(19) United States
(12) Patent Application Publication
(10) Pub. No.: US 2025/0352772 A1
(43) Pub. Date: Nov. 20, 2025

(54) NON-COMPLIANT MEDICAL BALLOON AND BALLOON CATHETER

(71) Applicant: OSYPKA AG, Rheinfelden-Baden (DE)

(72) Inventors: Wilfred Peter Heiner, Reinfelden (DE); Thorsten Gottsche, Rheinfelden (DE)

(73) Assignee: OSYPKA AG, Rheinfelden-Baden (DE)

(21) Appl. No.: 18/970,433

(22) Filed: Dec. 5, 2024

(30) Foreign Application Priority Data
May 14, 2024 (DE) 10 2024 113 408.9

(51) Int. Cl. A61M 25/10

(52) U.S. Cl. CPC A61M 25/10; A61M 2025/1075

(57) ABSTRACT

The invention relates to a non-compliant medical balloon for a balloon catheter having a base balloon and having a film reinforcement layer which comprises film made of a high-molecular polymer, in particular polyimide, LCP polymer, PET and/or PEEK, and surrounds the base balloon at least in sections.

### CROSS-REFERENCE TO RELATED APPLICATION

This application claims priority to German Patent Application No. 10 2024 113 408.9, filed on May 14, 2024, all of which is hereby incorporated by reference in its entirety.

### FIELD OF THE INVENTION

The invention relates to a non-compliant medical balloon for a balloon catheter and a balloon catheter having such a balloon.

### BACKGROUND OF THE INVENTION

In medicine, balloon catheters are catheters that are fitted with a balloon. The balloon can be expanded with compressed air or with a liquid, for example. The shape of the balloon catheter depends on the intended use in various medical specialties.

In angiology, for example, constricted or blocked blood vessels are widened using a balloon catheter. This is known as balloon dilatation. In urology, bladder catheters are used to drain urine from the bladder. However, balloon catheters are also used in orthopedics, pneumology and gynecology in a corresponding form.

In angiology in particular, non-compliance with such balloon catheters is especially important in order to avoid damage to the blood vessels during balloon dilatation.

Non-compliance refers to the property of the balloon not to expand further after reaching an expanded target shape when a nominal pressure is applied, i.e. to retain its shape and volume as the pressure in the balloon continues to increase. Non-compliant balloons can be described as non-yielding or non-expandable balloons and exert a high pressure to expand up to a certain diameter, but not significantly beyond. They can be used in angiology to expand occluded blood vessels, break up calcified lesions or expand stents.

The object of the invention is to provide a non-compliant medical balloon for a balloon catheter with improved usage properties and a balloon catheter with such a balloon.

### SUMMARY OF THE INVENTION

To solve this object, a non-compliant medical balloon for a balloon catheter having the features of independent claim 1 is first proposed. To solve the object, it is thus proposed in particular in the balloon mentioned at the beginning that the balloon comprises a base balloon and a film reinforcement layer, wherein the film reinforcement layer comprises film made of a high-molecular polymer and surrounds the base balloon at least in sections.

A balloon of this type offers particularly high resistance to additional expansion or stretching and can also meet strict non-compliance requirements. Here, the behavior of the balloon is considered that it exhibits when the internal pressure of the balloon is increased from the nominal pressure, for example the working pressure, to the burst pressure. Non-compliance is measured as a percentage change in diameter as a function of the applied pressure. For example, balloons that show a percentage change in diameter of less than two percent, preferably less than one percent, when the pressure changes from the nominal pressure to the burst pressure can be regarded as non-compliant.

The film reinforcement layer can comprise film made of polyimide, LCP polymer, PET and/or PEEK (polyether ether ketone), for example. The base balloon, which can be made of a semi-compliant material such as polyamide, is reinforced with the aid of the film reinforcement layer so that the medical balloon as a whole is non-compliant, i.e. non-yielding, due to the film reinforcement layer.

The film reinforcement layer can be attached at least indirectly to the base balloon. For example, the film reinforcement layer can be attached to the base balloon by means of an adhesive strip, preferably a polyimide adhesive strip.

The film reinforcement layer can comprise at least one film adhesive strip made of film made of a high molecular weight polymer, which is glued on to reinforce the base balloon. For example, a polyimide adhesive strip can be used as the film adhesive strip.

In a preferred embodiment of the balloon, the at least one film adhesive strip has a width of between 1 mm and 4 mm. Furthermore, the at least one film adhesive strip can have a film thickness of between 6 μm or 7.5 μm and 25 μm, in particular between 6 μm and 15 μm. It is also possible for the film of the film reinforcement layer, in particular the film adhesive strip, to have a bonding layer, in particular of thermoplastic polyurethane. This bonding layer can then be used to attach the film, in particular the film adhesive strip, directly to the base balloon of the balloon or also to an intermediate layer, for example a fiber-reinforcing layer, which will be explained in more detail below. The bonding layer can have a thickness of 15 μm to 40 μm, for example.

The film of the film reinforcement layer can consist of crystalline high-molecular polymer. It is also possible to use film made of amorphous high-molecular polymer or a mixture of amorphous and crystalline high-molecular polymer for the film reinforcement layer.

The film reinforcement layer can be wrapped around the base balloon. This is preferably over the entire surface in order to reinforce the base balloon in the best possible way and to achieve particularly high non-compliance of the balloon as a whole.

The film of the film reinforcement layer can, for example, have a film thickness of between 6 μm or 7.5 μm and 25 μm, for example between 6 μm and 15 μm. Preferably, the film thickness of the film reinforcement layer can be 15 μm.

The film reinforcement layer can be formed from at least one film section which is wrapped around the base balloon and comprises at least one film adhesive strip which is glued on to fix the film section.

In one embodiment of the balloon, the film reinforcement layer, in particular the at least one film adhesive strip and/or the at least one film section of the film reinforcement layer, is wrapped around the base balloon in at least one layer, but preferably in several, in particular two to five, layers. The number of layers of the film reinforcement layer can increase the non-compliance of the balloon. One layer of the film reinforcement layer can then consist of one film layer.

The balloon may include a fiber reinforcement layer. The fiber reinforcement layer may comprise high modulus polyethylene fibers or consist of high modulus polyethylene fibers. Suitable fibers include, for example, so-called Dyneema fibers. The fiber reinforcement layer may surround the base balloon at least in sections and promote non-compliance of the balloon as a whole.

In one embodiment of the balloon, the fiber reinforcement layer is arranged between the base balloon and the film reinforcement layer. The film reinforcement layer can, for example, be glued to the fiber reinforcement layer by means of at least one film adhesive strip.

In one embodiment of the balloon, the fiber reinforcement layer is designed as a knitted, warp-knitted and/or woven fabric. The fiber reinforcement layer serves to additionally reinforce the base balloon, which is already reinforced by the film reinforcement layer. In this way, a balloon can be provided that exhibits even greater non-compliance when the nominal pressure is exceeded.

Due to the film reinforcement layer provided according to the invention, the base balloon can be made of a material that is itself non-compliant, i.e. flexible or stretchable and therefore optionally more favorable. For example, the base balloon can be made of a semi-compliant material such as polyamide, polyamide 11, polyamide 12, polyamide made of m-xylylenediamine. The base balloon can, for example, be made of MX-nylon, a polyether block amide such as PEBAX and/or polyethylene or a mixture thereof.

In one embodiment of the balloon, it has at least one electronic component. The at least one electronic component may, for example, be a sensor and/or at least one electronic circuit and/or at least one sensor and/or at least one sensing pole and/or at least one stimulation pole.

The at least one electronic component can be arranged on an outer surface of the balloon, in particular on an outer surface of a conical section of the balloon. In this way, it is possible to bring the electronic component into contact with a target tissue when using the balloon in order to enable interaction of the electronic component with the target tissue.

The interaction of the electronic component with the target tissue then depends on the type of electronic component. A sensor or a sensing pole can be used to record parameters of interest, such as measured values and/or electrophysiological body signals, from the area of application of the balloon.

If the balloon has at least one stimulation pole as at least one electronic component, electromedical pulses can also be delivered to a target tissue via the stimulation pole if required.

In one embodiment of the balloon, it has at least one electronic component comprising at least one pressure sensor, at least one flow sensor and/or at least one ultrasonic sensor. With the aid of these sensors, it is possible to detect parameters of interest, for example from a patient's blood vessel when the balloon is used. Ultrasonic sensors are suitable, for example, for displaying differences in vessel structure and/or vessel density. This is relevant when the balloon is used in blood vessels.

If the balloon has a coil as an electronic component, it is possible to transfer energy inductively to the balloon. If the balloon has two coils, it is possible to carry out RF ablation with the balloon. Furthermore, it is also possible to transmit data and/or information via the coil either to the balloon and an electronic component arranged on it and/or from the balloon to the outside. Furthermore, a coil may also make it possible to improve the MR or X-ray visibility of the balloon in the patient's body.

The at least one electronic circuit and/or the at least one coil can be processed, for example printed and/or lithographed. In this way, it is possible to apply the circuit and/or the coil particularly efficiently and in a thin layer to a carrier surface, for example on the outside of the balloon.

The at least one electronic circuit and/or the at least one coil can be used as an antenna, in particular as an intravascular antenna. In this way, it is possible to use the balloon as an MRI antenna to visualize vessels to be examined, in particular blood vessels.

The balloon can also comprise an outer layer that surrounds the base balloon and at least one reinforcing layer, if present. The outer layer may consist of a thermoplastic material that has a lower modulus of elasticity than the material of the film reinforcement layer. The outer layer can, for example, consist of or comprise thermoplastic polyurethane and/or polyamide. The outer layer can serve to protect the parts of the balloon located inside the outer layer, for example the base balloon and/or at least the film reinforcement layer and/or at least one electronic component of the balloon. In addition, the outer layer may have favorable frictional properties and thus reduce friction between the balloon and the application area of the balloon, for example the inner wall of a blood vessel. This can simplify the application of the balloon during a catheter intervention.

As such, the outer layer can have a layered structure that preferably comprises a polyamide layer as the outer layer. Polyamide is characterized by a low coefficient of friction and is therefore particularly suitable as a friction-minimizing outer layer of the outer layer of the balloon.

The outer layer of the balloon can be designed as an outer balloon and/or consist of a semi-compliant material, for example polyamide, polyamide 11, polyamide 12, polyamide made of m-xylylenediamine, for example MX nylon, a polyether block amide, for example PEBAX, and/or polyethylene or a mixture thereof.

The individual layers of the balloon, for example the base balloon, the at least one reinforcement layer and/or the outer layer can be joined together, for example by lamination.

The balloon can comprise MR-visible and/or X-ray-visible, in particular metallic, particles. In this way, it is possible to visualize the balloon in the body using MR or X-ray techniques. This can considerably simplify the use of the balloon in a patient's body. The MR-visible and/or X-ray-visible particles can, for example, be arranged in the material of the base balloon, in the film reinforcement layer and/or in one or more of the fiber reinforcement layers and/or in an outer layer of the balloon.

Preferably, metal particles, for example iron particles and/or dysprosium particles and/or dysprosium oxide particles, are used as MR-visible and/or X-ray-visible particles.

The balloon may have at least one MR-visible and/or X-ray-visible marker. This can also promote the MR and/or X-ray visibility of the balloon. With the aid of such a marker or also the aforementioned MR-visible and/or X-ray-visible particles, the position, shape and/or orientation of the balloon can be made recognizable with imaging systems.

Finally, to solve the object, a balloon catheter with a balloon according to one of the claims directed to such a balloon is also proposed.

The invention is described in more detail below with reference to an exemplary embodiment, but is not limited to this exemplary embodiment. Further exemplary embodiments are obtained by combining the features of individual or several claims with one another and/or by combining individual or several features of the exemplary embodiment, wherein:

### BRIEF DESCRIPTION OF THE DRAWINGS

FIG. 1 shows a perspective view of a non-compliant medical balloon according to the invention,

FIG. 2 shows a side view of a base balloon of the balloon shown in FIG. 1,

FIG. 3 shows a side view of the base balloon shown in FIG. 2 after a fiber reinforcement layer of high modulus polyethylene fibers has been attached,

FIG. 4 shows a side view of the base balloon shown in FIG. 3 after application of a film reinforcement layer,

FIG. 5 shows a side view of the base balloon shown in FIG. 4 after application of an outer layer of a thermoplastic polyurethane, and

FIG. 6 shows a side view of a non-compliant balloon which has a base balloon and a film reinforcement layer, wherein the film reinforcement layer comprises film made of a high molecular weight polymer, preferably polyimide, and surrounds the base balloon at least in sections, and wherein the film reinforcement layer is bonded directly to the base balloon so that no fiber reinforcement layer is arranged between the base balloon and the film reinforcement layer.

All figures show at least parts of a non-compliant or non-yielding balloon designated as a whole as 1. The balloon 1 comprises a base balloon 2, which is shown in FIG. 2.

### DETAILED DESCRIPTION

The base balloon 2 can be made of a material that is itself only semi-compliant, for example polyamide, polyamide 11, polyamide 12, polyamide made of m-xylylenediamine, for example MX nylon, a polyether block amide, for example PEBAX, and/or polyethylene or a mixture thereof.

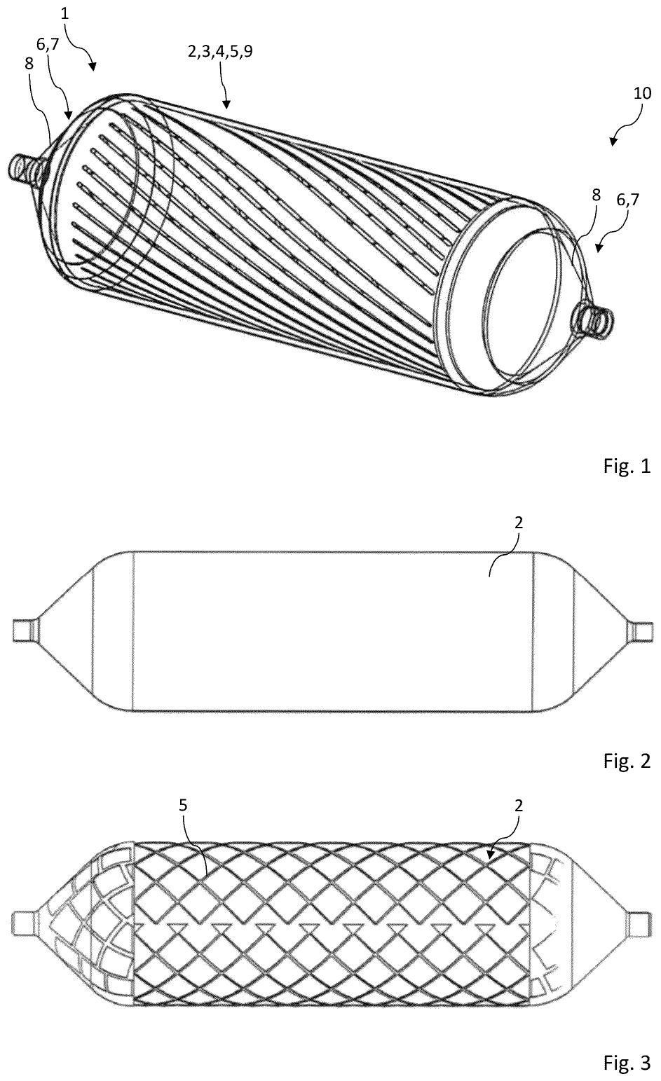

The balloon 1 shown in FIGS. 1 to 5 comprises two reinforcement layers 3 and 5, which surround the base balloon 2 at least in sections and promote a particularly high non-compliance of the balloon 1.

The first reinforcement layer is shown in FIG. 3 and is designed as a fiber reinforcement layer 5. The fiber reinforcement layer 5 is formed from high-modulus polyethylene fibers, for example Dyneema fibers. The fiber reinforcement layer 5 can be formed as a knitted fabric, warp-knitted fabric and/or woven fabric.

As a further and particularly important reinforcement layer, the balloon 1 has a film reinforcement layer 3 surrounding the fiber reinforcement layer 5 and the base balloon 2 underneath, which comprises polyimide film, specifically polyimide film adhesive strips 4. The film reinforcement layer 3 reinforces the balloon 1 and helps to ensure that the balloon 1 does not expand further once the nominal pressure has been exceeded.

The film reinforcement layer 3 thus comprises at least one film adhesive strip 4 made of film made of a high molecular weight polymer, in this case a polyimide film adhesive strip, which is glued on to reinforce the base balloon 2.

The film of the film reinforcement layer 3 consists of crystalline and/or amorphous high-molecular polymer.

The figures illustrate that the film reinforcement layer 3 is wrapped around the base balloon 2. The film of the film reinforcement layer 3 can, for example, have a film thickness of between 6 μm and 25 μm or 7.5 μm and 25 μm, preferably between 6 μm and 15 μm.

The fiber reinforcement layer 5 is arranged between the base balloon 2 and the film reinforcement layer 3. The film reinforcement layer 3 and its adhesive film strips 4 are glued to the fiber reinforcement layer 5. The film adhesive strips 4 run diagonally to the longitudinal central axis of the balloon 1 and the base balloon 2 and may, for example, have a bonding layer of thermoplastic polyurethane. The bonding layer can have a thickness of 15 μm to 40 μm, for example.

The film of the film reinforcement layer 3 can also be made of a high-molecular polymer other than polyimide, for example LCP polymer, PET and/or PEEK (polyether ether ketone).

The balloon 1 can have at least one electronic component 6, for example a sensor, at least one electronic circuit, at least one coil, at least one sensing pole and/or at least one stimulation pole.

The at least one electronic component 6 can be arranged on an outer surface 7 of the balloon 1, for example on an outer surface 7 of a conical section 8 of the balloon 1.

The balloon 1 can have at least one electronic component 6, for example a pressure sensor, at least one flow sensor and/or at least one ultrasonic sensor. If the electronic component 6 of the balloon 1 is an electronic circuit and/or a coil, these can be processed, for example printed and/or lithographed.

The at least one electronic circuit and/or the at least one coil can be used as an antenna, for example as an intravascular antenna, which can be brought into a target area to be examined and/or treated in the patient's body with the aid of the balloon 1.

FIG. 5 shows that the balloon 1 comprises an outer layer 9 which surrounds the base balloon 2 and the reinforcement layers 4 and 5. The outer layer 9 of the balloon 1 shown in FIG. 5 can also be referred to as the outer balloon. The outer layer 9 consists of a thermoplastic material which has a lower modulus of elasticity than the material of the base balloon 2. Preferably, the outer layer 9 is made of thermoplastic polyurethane and/or polyamide. The outer layer 9 may have a layered structure, wherein the outer layer is preferably a polyamide layer which provides the balloon 1 with favorable frictional properties for its use in a blood vessel. The outer layer 9 can be made of the same material as the base balloon 2.

The balloon 1 can have, for example in the base balloon 2, in the reinforcement layer 4 and/or 5 and/or in the outer layer 9, MR-visible and/or X-ray-visible, in particular metallic, particles. These particles can be, for example, iron particles and/or dysprosium particles and/or dysprosium oxide particles. With the aid of these particles, it is possible to visualize the balloon 1 in the body of a patient during the application of the balloon 1 with the aid of corresponding imaging systems and methods based on MR or X-ray technology.

The balloon 1 can have an MR-visible and/or X-ray-visible marker instead of or in addition to the particles. Such a marker can be understood as a structure that can be made visible by means of an MR and/or X-ray method.

The balloon 1 can be used as part of a balloon catheter shown as a whole with 10 and only strongly schematized.

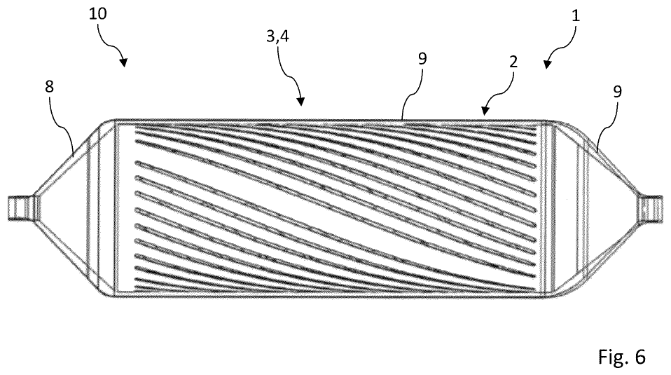

The balloon 1 shown in FIG. 6 is similar to the balloon 1 shown in FIGS. 1 to 5 and has a film reinforcement layer 3, but no fiber reinforcement layer 4. The film reinforcement layer 3 can comprise several film layers. The film layers can be formed by adhesive film strips 4 and/or film sections that are wrapped around the base balloon 2 and attached to it. The non-compliance of the balloon 1 is increased by several film layers. In the exemplary embodiment of the balloon 1 shown in FIG. 6, the film reinforcement layer 3 also comprises film made of a high molecular weight polymer, in particular polyimide, LCP polymer, PET and/or PEEK.

The balloon 1 shown in FIG. 6 has a film reinforcement layer 3, which specifically consists of several adhesive film strips 4. The film adhesive strips 4 each have a width of between 1 mm and 4 mm and a film thickness of between 6 μm and 25 μm, in particular between 6 μm and 15 μm.

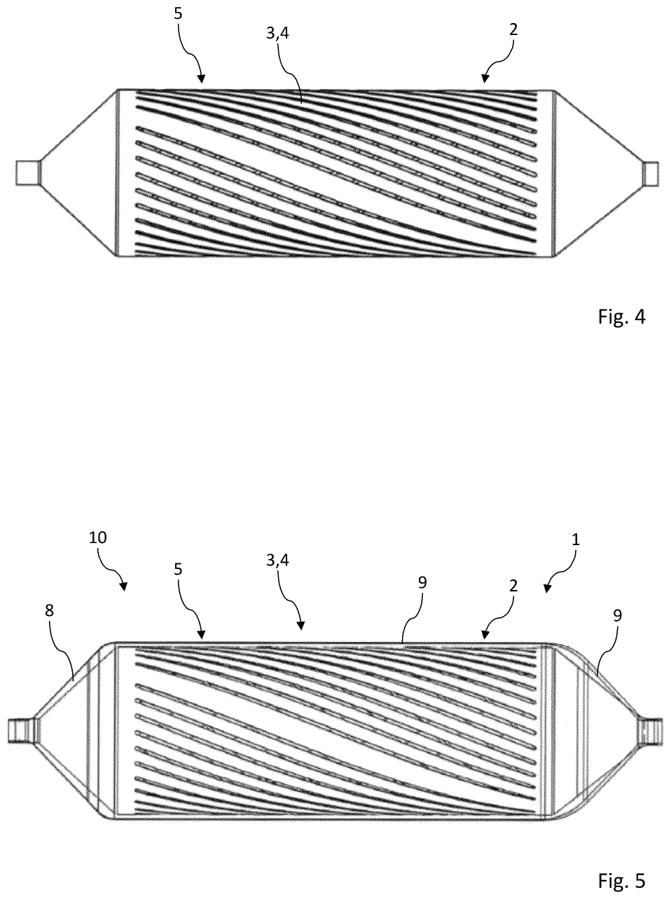

Each film adhesive strip 4 has a bonding layer on its side facing the base balloon 2, which consists of thermoplastic polyurethane (TPU) and has a layer thickness of 15 μm to 40 μm. The film adhesive strips 4 are wrapped around the base balloon 2 in one to five layers and, as also shown for the balloon 1 in FIG. 4, in a pattern diagonal to a longitudinal central axis of the balloon 1 and the base balloon 2 and are fixed directly to the base balloon 2 via their connecting layer of thermoplastic polyurethane.

The balloon 1 shown in FIG. 6 also has an outer layer 9. Like the base balloon 2, the outer layer 9 consists of a semi-compliant material, for example polyamide, polyamide 11, polyamide 12, polyamide made of m-xylylenediamine, for example MX nylon, a polyether block amide, for example PEBAX, and/or polyethylene or a mixture thereof. The outer layer 9 can also be referred to as an outer balloon.

1. A non-compliant medical balloon for a balloon catheter having a base balloon and having a film reinforcement layer which comprises film made of a high-molecular polymer, including polyimide, LCP polymer, PET and/or PEEK, and surrounds the base balloon at least in sections,
wherein the film reinforcement layer is wrapped around the base ballon,
wherein the balloon comprises a fiber reinforcement layer surrounding the base balloon at least in sections,
wherein the fiber reinforcement layer is arranged between the base balloon and the film reinforcement layer, and
wherein the fiber reinforcement layer is formed as a knitted fabric, warp-knitted fabric and/or woven fabric.

2. The balloon of claim 1, wherein the film reinforcement layer is attached at least indirectly to the base balloon, by means of a polyimide adhesive strip.

3. The balloon of claim 1, wherein the film reinforcement layer comprises at least one film adhesive strip of film made of a high molecular weight polymer, which is glued on to reinforce the base balloon.

4. The balloon of claim 3, wherein the at least one film adhesive strip has a width of between 1 mm and 4 mm and/or a film thickness of between 6 μm or 7.5 μm and 25 μm, and/or a bonding layer, of thermoplastic polyurethane, with a layer thickness of 15 μm to 40 μm.

5. The balloon of claim 1, wherein the film of the film reinforcement layer consists of crystalline and/or amorphous high-molecular polymer.

6. (canceled)

7. The balloon of claim 1, wherein the film of the film reinforcement layer has a film thickness between 6 μm or 7.5 μm and 25 μm.

8. The balloon of claim 1, wherein the film reinforcement layer comprises at least one film section wrapped around the base balloon and at least one film adhesive strip which is glued on to fix the film section.

9. The balloon of claim 1, wherein the at least one film adhesive strip and/or the at least one film section, is wrapped around the base balloon in at least one layer.

10. (canceled)

11. (canceled)

12. The balloon of claim 10, wherein the fiber reinforcement layer comprises high modulus polyethylene fibers or consists of high modulus polyethylene fibers.

13. (canceled)

14. The balloon of claim 1, wherein the base balloon is made of a semi-compliant material.

15. The balloon of claim 1, wherein the balloon comprises at least one electronic component, including a sensor, and/or at least one electronic circuit and/or at least one coil and/or at least one sensing pole and/or at least one stimulation pole, which is/are arranged, on and/or in the film reinforcement layer.

16. The balloon of claim 15, wherein the at least one electronic component is arranged on an outer surface of the balloon.

17. The balloon of claim 15, wherein the balloon comprises the at least one electronic component, at least one pressure sensor, at least one flow sensor, and/or at least one ultrasonic sensor.

18. The balloon of claim 15, wherein the at least one electronic circuit and/or the at least one coil is/are processed, on the film of the film reinforcement layer.

19. The balloon of claim 15, wherein the at least one electronic circuit and/or the at least one coil can be used as an intravascular antenna.

20. The balloon of claim 1, wherein the balloon comprises an outer layer that surrounds the base balloon, wherein the outer layer is made of a, thermoplastic material having a lower modulus of elasticity than the material of the film reinforcement layer, wherein the outer layer consists of or comprises, thermoplastic, polyurethane and/or polyamide.

21. The balloon of claim 20, wherein the outer layer has a layered structure, comprising a polyamide layer as the outer layer.

22. The balloon of claim 1, wherein the base balloon and/or the film reinforcement layer and/or one or the fiber reinforcement layer and/or one or the outer layer, comprises MR-visible and/or X-ray-visible, metallic particles, including iron particles and/or dysprosium particles and/or dysprosium oxide particles.

23. The balloon of claim 1, wherein the balloon comprises at least one MR-visible and/or X-ray-visible marker.

24. (canceled)
